(19) United States
(12) Patent Application Publication
(10) Pub. No.: US 2021/0354343 A1
(43) Pub. Date: Nov. 18, 2021

(54) APPARATUS FOR MANUFACTURING MICRONEEDLE PATCH

(71) Applicant: SNVIA Co., Ltd., Busan (KR)

(72) Inventors: Kang Oh LEE, Busan (KR); Seung Soo LEE, Busan (KR); Seung Yun YANG, Daegu (KR); Keum-Yong SEONG, Busan (KR); Sodam KIM, Busan (KR)

(73) Assignee: SNVIA Co., Ltd., Busan (KR)

(21) Appl. No.: 16/876,730

(22) Filed: May 18, 2020

(51) Int. Cl. B29C 43/02; A61M 37/00; B29C 43/36

(52) U.S. Cl. CPC B29C 43/021; A61M 37/0015; B29C 2043/3222; A61M 2037/0053; B29C 2043/026; B29C 43/36

(57) ABSTRACT

Disclosed is an apparatus for manufacturing a microneedle patch, including: a silicon mold mounted on an upper surface to mold a material for the microneedle patch; a carrier of which the silicon mold is mounted on an inside of the upper surface; an upper block which is assembled with the carrier to form a chamber in which the material and the silicon mold are accommodated; an air cylinder which is connected to the upper surface of the upper block to transmit power so that the upper block descends to be assembled with the carrier and ascends to be separated from the carrier; and a pressing portion which is connected to a side surface of the upper block so as to press the chamber or ventilate the chamber while the upper block and the carrier are assembled with each other.

### BACKGROUND

The present invention relates to an apparatus for manufacturing a microneedle patch and more particularly, to an apparatus for manufacturing a microneedle patch capable of removing microbubbles generated in a manufacturing process of the microneedle patch.

In manufacturing of a microneedle patch, there is a problem in that microbubbles are generated in a silicon mold, and thus, in order to solve the problem, there is a method of removing the microbubbles by inflating the bubbles by applying a vacuum pressure to float on the surface of the silicon mold.

However, in this way, when the inflated bubbles are attached to an apex of the silicone mold when floating, there is a disadvantage that it takes a lot of time for the bubbles to fall by buoyancy.

In addition, as can be seen in FIG. 1, when the vacuum pressure is applied, as there is a phenomenon in which dissolved oxygen melted in a chemical material is eluted, there is a problem that a product is dried in a state in which the microbubbles remain.

In addition, the bubbles were also removed by a pressing method using a device such as a press or a roller, but even in this case, there is a second contamination problem due to a contact of the chemical material with the pressing device, and there is also a problem that the contact adversely affects the cleanliness of the manufacturing process.

### PRIOR ART

(Patent Document 1) U.S. Pat. No. 8,834,423 (Sep. 16, 2014)

(Patent Document 2) U.S. Pat. No. 8,353,861 (Jan. 15, 2013)

### SUMMARY

The present invention is to solve the above-mentioned problems of the prior art, and relates to an apparatus for manufacturing a microneedle patch capable of effectively removing microbubbles generated in a manufacturing process of the microneedle patch.

In addition, the present invention relates to an apparatus for manufacturing a microneedle patch capable of increasing productivity by further shortening a manufacturing time of the microneedle patch.

Further, the present invention relates to an apparatus for manufacturing a microneedle patch capable of minimizing contamination of the microneedle patch with a chemical material.

The technical objects to be achieved by the present invention are not limited to the aforementioned technical objects, and other technical objects, which are not mentioned above, will be apparently appreciated by a person having ordinary skill in the art from the following description.

An apparatus for manufacturing a microneedle patch according to the present invention to be proposed as described above comprises a silicon mold mounted on an upper surface to mold a material for the microneedle patch; a carrier of which the silicon mold is mounted on an inside of the upper surface; an upper block which is assembled with the carrier to form a chamber in which the material and the silicon mold are accommodated; an air cylinder which is connected to the upper surface of the upper block to transmit power so that the upper block descends to be assembled with the carrier and ascends to be separated from the carrier; and a pressing portion which is connected to a side surface of the upper block so as to press the chamber or ventilate the chamber while the upper block and the carrier are assembled with each other, wherein after the pressing and the ventilating are performed by the pressing portion while the upper block descends to be assembled with the carrier to form the chamber together with the carrier, the upper block ascends to be separated from the carrier, and the pressing and the ventilating are repeated many times by the pressing portion so that the bubbles introduced into the material are removed in the molding process of the material.

According to the apparatus for manufacturing the microneedle patch by the present invention as described above, since the molding of the material including the chemical material is performed by repeating the pressing and the ventilation many times by introducing the gas while the material and the chemical material for manufacturing the microneedle patch are mounted on the silicon mold, there is an advantage of effectively removing the microbubbles generated in the manufacturing process of the microneedle patch through the repeated pressing and ventilation processes.

In addition, as compared to a conventional method of removing bubbles by a vacuum pressure, according to the present invention, it is possible to increase productivity by further shortening the manufacturing time of the microneedle patch.

Further, as compared to a conventional method of directly pressing the material including the chemical material with a device such as a press or a roller, according to the present invention, since the pressing is performed by gas such as air without a direct contact by the device, there is an advantage of minimizing the contamination of the microneedle patch with the chemical material.

### BRIEF DESCRIPTION OF THE DRAWINGS

FIG. 1 is a photograph showing a state in which bubbles are removed by using a vacuum pressure in the related art.

FIG. 2 is a perspective view showing an apparatus for manufacturing a microneedle patch according to the present invention.

FIG. 3 is a front view showing a state in which an upper block ascends in the apparatus for manufacturing the microneedle patch according to the present invention.

FIG. 4 is a front view showing a state in which the upper block descends in the apparatus for manufacturing the microneedle patch according to the present invention.

FIG. 5 is a view showing a state of the upper block in the apparatus for manufacturing the microneedle patch according to the present invention.

FIG. 6 is a view showing a process in which pressing and ventilation are repeated by a pressing portion in the apparatus for manufacturing the microneedle patch according to the present invention.

FIG. 7 is a view showing a state in which a carrier, a silicon mold, and a sealing member are coupled to each other in the apparatus for manufacturing the microneedle patch according to the present invention.

FIG. 8 is a view showing a state in which the carrier, the silicon mold, and the sealing member are separated from each other in the apparatus for manufacturing the microneedle patch according to the present invention.

FIG. 9 is a view showing a detailed state of the carrier in the apparatus for manufacturing the microneedle patch according to the present invention.

FIG. 10 is a view showing a state in which a continuous pressing process is performed by disposing a plurality of apparatuses for manufacturing the microneedle patch according to the present invention.

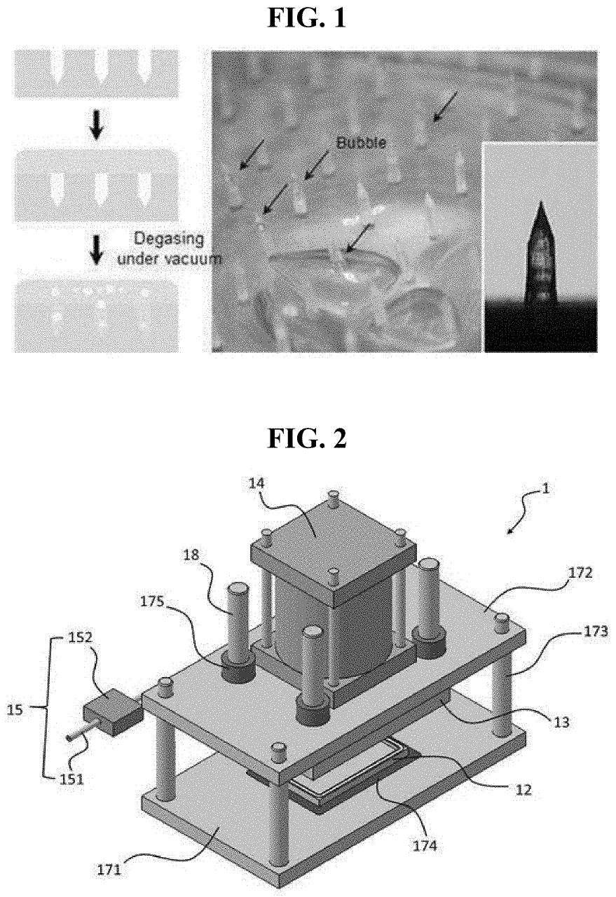

### DETAILED DESCRIPTION

Hereinafter, an embodiment of an apparatus for manufacturing a microneedle patch according to the present invention will be described in detail with reference to the accompanying drawings.

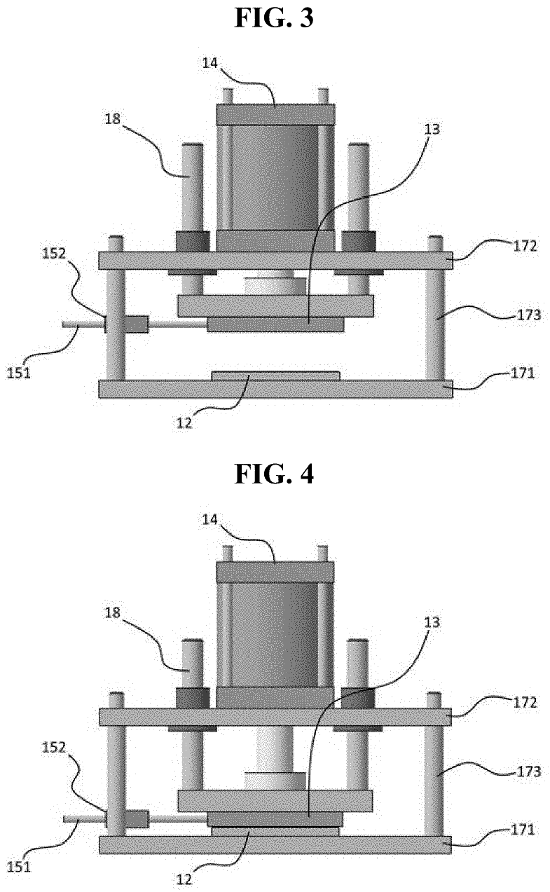

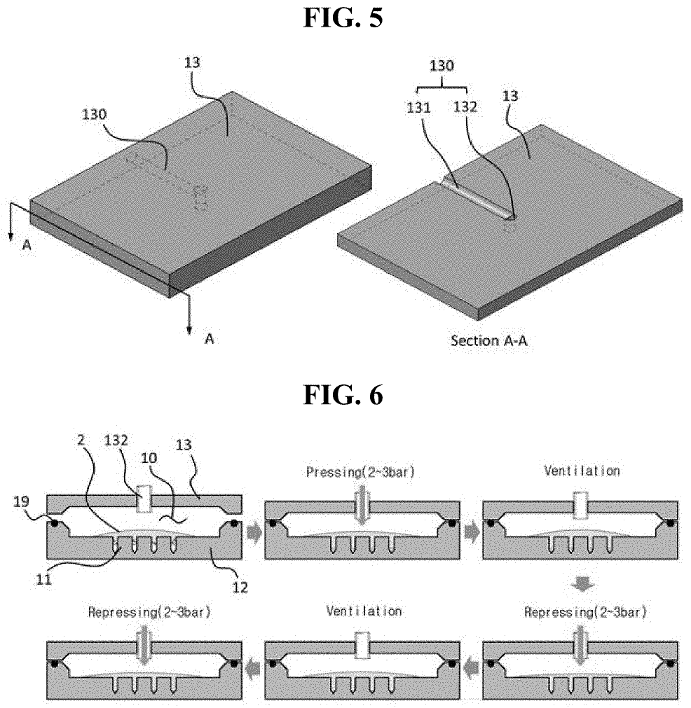

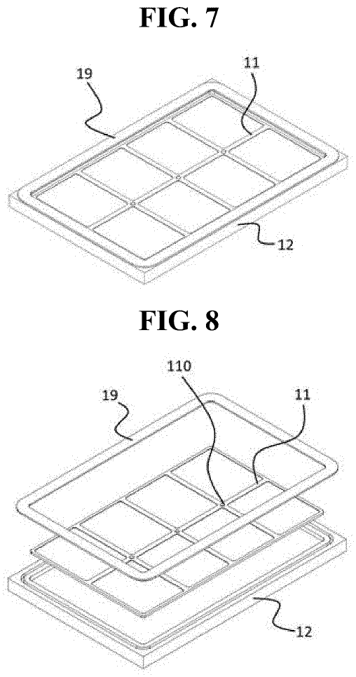

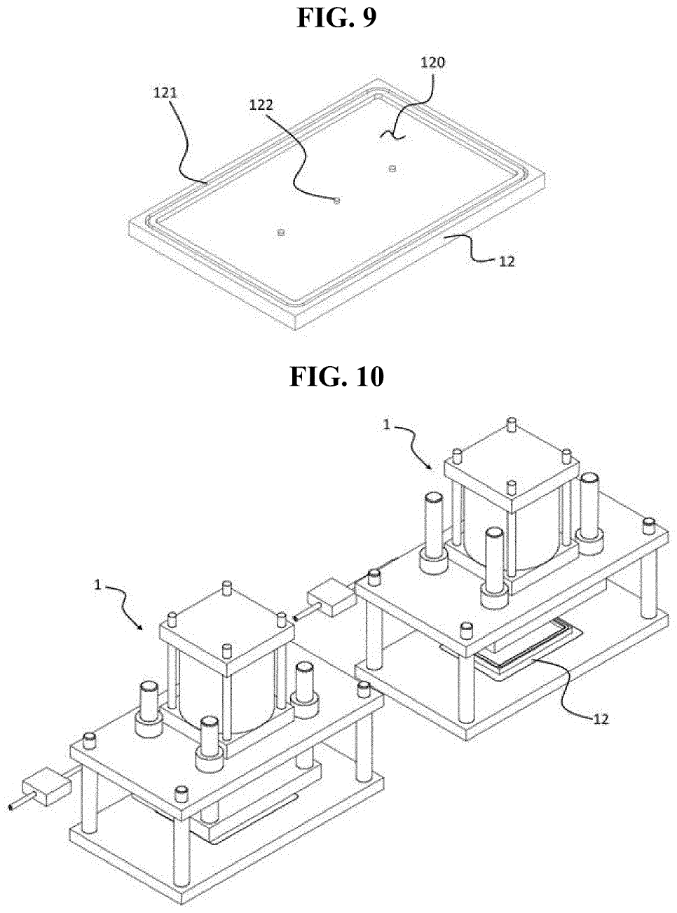

FIG. 2 is a perspective view showing an apparatus for manufacturing a microneedle patch according to the present invention and FIG. 3 is a front view showing a state in which an upper block ascends in the apparatus for manufacturing the microneedle patch according to the present invention. FIG. 4 is a front view showing a state in which the upper block descends in the apparatus for manufacturing the microneedle patch according to the present invention, FIG. 5 is a view showing a state of the upper block in the apparatus for manufacturing the microneedle patch according to the present invention, and FIG. 6 is a view showing a process in which pressing and ventilation are repeated by a pressing portion in the apparatus for manufacturing the microneedle patch according to the present invention. FIG. 7 is a view showing a state in which a carrier, a silicon mold, and a sealing member are coupled to each other in the apparatus for manufacturing the microneedle patch according to the present invention, FIG. 8 is a view showing a state in which the carrier, the silicon mold, and the sealing member are separated from each other in the apparatus for manufacturing the microneedle patch according to the present invention, FIG. 9 is a view showing a detailed state of the carrier in the apparatus for manufacturing the microneedle patch according to the present invention, and FIG. 10 is a view showing a state in which a continuous pressing process is performed by disposing a plurality of apparatuses for manufacturing the microneedle patch according to the present invention.

Referring to FIGS. 2 to 10, an apparatus 1 for manufacturing a microneedle patch according to the present invention includes a silicon mold 11 mounted on an upper surface to mold a material 2 for the microneedle patch, a carrier 12 of which the silicon mold 11 is mounted on an inside of the upper surface, an upper block 13 which is assembled with the carrier 12 to form a chamber 10 in which the material 2 and the silicon mold 11 may be accommodated, an air cylinder 14 which is connected to the upper surface of the upper block 13 to transmit power so that the upper block 13 may descend to be assembled with the carrier 12 and may ascend to be separated from the carrier 12, and a pressing portion 15 which is connected to a side surface of the upper block 13 so as to press the chamber 10 or ventilate the chamber 10 while the upper block 13 and the carrier 12 are assembled with each other.

The carrier 12 is provided to be transferable so that the position is variable. While the carrier 12 is transferred so as to be positioned directly below the upper block 13 and the upper block 13 descends to be assembled with the carrier 12, the pressing and the ventilation are performed by the pressing portion 15. In addition, after the pressing and the ventilation are completed, the upper block 13 ascends to be separated from the carrier 12 and the carrier 12 may be transferred to be detached from a directly lower side of the upper block 13.

The apparatus 1 for manufacturing the microneedle patch further includes a base plate 171 on which the carrier 13 is transferred and mounted, a separation plate 172 which is positioned to be spaced apart from an upper side of the base plate 171 to have a space formed between the base plate 171 and the separation plate 172 so that the carrier 12 and the upper block 13 may be transferred, a supporter 173 which connects the base plate 171 and the separation plate 172 so that the separation plate 172 may be fixed while being spaced apart from the base plate 171, and a guide post 18 which is provided so as to ascend vertically by passing through a guide hole 175 formed in the separation plate 172 and has a lower end fixed to the upper surface of the upper block 13 to guide an ascending direction of the upper block 13.

The base plate 171 has a quadrangular planar shape and serves to support the remaining portion of the apparatus 1 for manufacturing the microneedle patch. A heating plate 174 for heating the carrier 12 and the silicon mold 11 is installed at the center of the upper surface of the base plate 171, that is, a point where the carrier 12 is positioned.

The separation plate 172 is also formed in the same quadrangular planar shape as the base plate 171, and serves to support the air cylinder 14, the guide post 18, the upper block 13, and the like.

In addition, in the apparatus 1 for manufacturing the microneedle patch, while the carrier 12 is transferred directly below the upper block 13 and the upper block 13 descends to be assembled with the carrier 12 so as to form the chamber 10 together with the carrier 12, after the pressing and the ventilation are performed by the pressing portion 15, the upper block 13 ascends to be separated from the carrier 12 and the carrier 12 is detached from the directly lower portion of the upper block 13 to mold the material 2. Further, in the molding of the material 2, the pressing and the ventilation are repetitively performed multiple times by the pressing portion 15 so that the bubbles introduced into the material 2 may be removed.

More specifically, the carrier 12 may be transferred and positioned to the upper surface of the base plate 171. That is, the carrier 12 is transferred by a driving device such as a conveyer to be positioned on the upper surface of the base plate 172, and after the molding of the material 2 is completed, the carrier 12 may be transferred to be detached from the base plate 171.

In addition, the silicon mold 11 is mounted on the upper surface of the carrier 12, which is transferred to the upper surface of the base plate 171, and the material 2 including a chemical material is mounted on the silicon mold 11 for manufacturing the microneedle patch.

The upper block 13 is positioned at the top while the carrier 12 is transferred, and then descends when the carrier 12 is positioned on the upper surface of the base plate 171, that is, directly below the upper block 13 to be assembled with the carrier 12 to form the chamber 10 together with the carrier 12.

At this time, a sealing member 19 is installed on an upper edge of the carrier 12 so that the chamber 10 may be firmly sealed while the carrier 12 is assembled with the upper block 13. In addition, a state in which the silicon mold 11 and the sealing member 19 are mounted on the upper surface of the carrier 12 is as shown in FIGS. 7 to 8.

In addition, as shown in FIG. 9, the center of the upper surface of the carrier 12 is formed with a wide and shallow mold installation groove 120 so that the silicon mold 11 may be mounted, and the edge of the upper surface of the carrier 12 is formed with a sealing groove 121 on which the sealing member 19 may be mounted and pressed so that airtightness may be reinforced. In addition, a fixing protrusion 122 is formed on the mold installation groove 120 of the carrier 12 so that the silicon mold 11 may be fixed more firmly. That is, while the silicon mold 11 is installed on the upper surface of the carrier 12, the fixing protrusion 122 is inserted into the fixing hole 110 formed in the silicon mold 11, so that the silicon mold 11 may be installed more firmly on the carrier 12.

Next, while the upper block 13 and the carrier 12 are assembled with each other to shield the chamber 10, gases such as air are introduced into the chamber 10 by the pressing portion 15 connected to the upper block 13 so that the pressure in the chamber 10 is increased.

At this time, in the upper block 13, a pressing flow path 130 communicating the pressing portion 15 and the chamber 10 is formed so that the gas supplied from the pressing portion 15 for pressing may be introduced into the chamber 10. Further, the pressing flow path 130 includes a horizontal flow path 131 which is extended from the side surface of the upper block 13 to the center of the upper block 13 to guide the gas introduced from the pressing portion 15 to the center of the upper block 13, and a vertical flow path 132 which is formed to be extended downward from the horizontal flow path 131 to the chamber 10 to guide the gas introduced into the horizontal flow path 131 to the chamber 10. Accordingly, the gas introduced into the upper block 13 is supplied to the chamber 10 through the pressing flow path 130 by the pressing portion 15, so that the pressure of the chamber 10 may be increased.

In addition, the pressing portion 15 includes a pressing pipe 151 which is connected to the upper block 13 to guide the gas to be supplied to the upper block 13 and a valve 152 which is installed at one side of the pressing pipe 151 to adjust a flow of the gas through the pressing pipe 151.

When the pressure of the chamber 10 is increased by the pressing portion 15, the material 2 and the silicon mold 11 are pressed by the gas pressure in the chamber 10, and microbubbles included in the material 2 are naturally detached and removed from the material 2. After the microbubbles of the material 2 are removed to some extent, high pressure gas in the chamber 10 is exhausted to the outside by the pressing portion 15, that is, the ventilation process is performed, so that the inner pressure of the chamber 10 is decreased.

The pressing and ventilation processes by the pressing portion 15 are repeatedly performed in the order of first pressing, first ventilation, second pressing, second ventilation, and third pressing. At this time, at the time of the first pressing, the second pressing, and the third pressing, the inner pressure of the chamber 10 is pressed to reach 2 to 3 bar.

The number of repetitions of the pressing and ventilation processes and the pressure value of the chamber 10 during pressing are obtained as a result of several experiments to find appropriate conditions capable of effectively removing the microbubbles included in the material 2. Accordingly, this can be seen as an optimal pressing condition in removing the microbubbles included in the material 2.

After the pressing and ventilation processes are completed, that is, the molding of the material 2 is completed, while the upper block 13 ascends, the chamber 10 is opened and the carrier 12 is transferred so that the molded material 2 may be discharged from the base plate 171.

Meanwhile, as shown in FIG. 10, a plurality of apparatuses 1 for manufacturing the microneedle patch is disposed in a conveyer line to be continuously transferred so that the pressing and ventilation processes may be repeated more times while the carrier 12 transferred along the conveyer passes sequentially through the plurality of apparatuses 1 for manufacturing the microneedle patch. Due to this, there is an advantage that the bubbles remaining in the silicon mold 11 and the material 2 may be more completely removed.

According to the present invention, since the molding of the material 2 including the chemical material is performed by repeating the pressing and the ventilation many times by introducing the gas while the material 2 and the chemical material for manufacturing the microneedle patch are mounted on the silicon mold 11, there is an advantage of effectively removing the microbubbles generated in the manufacturing process of the microneedle patch through the repeated pressing and ventilation processes.

Particularly, the pressing and ventilation processes are repetitively performed in the order of the first pressing, the first ventilation, the second pressing, the second ventilation, and the third pressing and under an optimal condition so that the inner pressure of the chamber 10 may reach 2 to 3 bar at the time of the first pressing, the second pressing, and the third pressing, thereby further maximizing an effect of removing the microbubbles.

In addition, as compared to a conventional method of removing bubbles by a vacuum pressure, according to the present invention, it is possible to increase productivity by further shortening the manufacturing time of the microneedle patch.

In addition, as compared to a conventional method of directly pressing the material 2 containing the chemical material with a device such as a press or a roller, according to the present invention, since the pressing is performed by gas such as air without a direct contact by the device, there is an advantage of minimizing the contamination of the microneedle patch with the chemical material.

As described above, within the scope of the basic technical idea of the present invention, many other modifications are enabled to those skilled in the art, and the scope of the present invention should be interpreted based on the appended claims.

[Explanation of Reference Numerals and Symbols]

| | |
|---|---|
| 1: Apparatus for manufacturing microneedle | 10: Chamber |
| patch | |
| 11: Silicon mold | 12: Carrier |
| 13: Upper block | 14: Air cylinder |
| 15: Pressing portion | 18: Guide post |
| 19: Sealing member | 110: Fixing hole |
| 120: Mold installation groove | 121: Sealing groove |
| 122: Fixing protrusion | 130: Pressing flow path |
| 131: Horizontal flow path | 132: Vertical flow path |
| 151: Pressing pipe | 152: Valve |
| 171: Base plate | 172: Separation plate |

What is claimed is:

1. An apparatus for manufacturing a microneedle patch comprising:
a silicon mold mounted on an upper surface to mold a material for the microneedle patch;
a carrier of which the silicon mold is mounted on an inside of the upper surface;
an upper block which is assembled with the carrier to form a chamber in which the material and the silicon mold are accommodated;
an air cylinder which is connected to the upper surface of the upper block to transmit power so that the upper block descends to be assembled with the carrier and ascends to be separated from the carrier; and
a pressing portion which is connected to a side surface of the upper block so as to press the chamber or ventilate the chamber while the upper block and the carrier are assembled with each other,
wherein after the pressing and the ventilation are performed by the pressing portion while the upper block descends to be assembled with the carrier to form the chamber together with the carrier, the upper block ascends to be separated from the carrier, and
the pressing and the ventilation are repeated many times by the pressing portion so that the bubbles introduced into the material are removed in the molding process of the material.

2. The apparatus for manufacturing the microneedle patch of claim 1, wherein the carrier is provided to be transferable so that the position is variable,
the pressing and the ventilation are performed by the pressing portion while the carrier is transferred so as to be positioned directly below the upper block and the upper block descends to be assembled with the carrier, and
after the pressing and the ventilation are completed, the upper block ascends to be separated from the carrier and the carrier is transferred to be detached from a directly lower side of the upper block.

3. The apparatus for manufacturing the microneedle patch of claim 2, wherein the pressing and ventilation processes by the pressing portion are repeatedly performed in the order of first pressing, first ventilation, second pressing, second ventilation, and third pressing, and
at the time of the first pressing, the second pressing, and the third pressing, the inner pressure of the chamber is pressed to reach 2 to 3 bar.

4. The apparatus for manufacturing the microneedle patch of claim 3, further comprising:
a sealing member which is installed on an edge of the upper surface of a lower block so that the chamber is firmly sealed while the lower block is assembled with the upper block.

5. The apparatus for manufacturing the microneedle patch of claim 4, wherein the upper block is formed with a pressing flow path communicating the pressing portion and the chamber so that gas supplied from the pressing portion for pressing is introduced into the chamber, and
the pressing flow path includes a horizontal flow path which is extended from the side surface of the upper block to the center of the upper block to guide the gas introduced from the pressing portion to the center of the upper block; and
a vertical flow path which is formed to be extended downward from the horizontal flow path to the chamber to guide the gas introduced into the horizontal flow path to the chamber.

6. The apparatus for manufacturing the microneedle patch of claim 5, further comprising:
a base plate on which the carrier is transferred and mounted;
a separation plate which is positioned to be spaced from an upper side of the base plate to have a space formed between the base plate and the separation plate so that the carrier, the lower block, and the upper block are transferred;
a supporter which connects the base plate and the separation plate so that the separation plate is fixed while being spaced apart from the base plate; and
a guide post which is provided so as to ascend vertically by passing through a guide hole formed in the separation plate and has a lower end fixed to the upper surface of the upper block to guide an ascending direction of the upper block.
